(19) United States
(12) Patent Application Publication
(10) Pub. No.: US 2021/0298639 A1
(43) Pub. Date: Sep. 30, 2021

(54) Apparatus for Breath Sample Collection

(71) Applicant: Joseph Zhili Huang, Monroe, NJ (US)

(72) Inventor: Joseph Zhili Huang, Monroe, NJ (US)

(21) Appl. No.: 17/214,907

(22) Filed: Mar. 28, 2021

Related U.S. Application Data
(60) Provisional application No. 63/001,103, filed on Mar. 27, 2020.

(51) Int. Cl. A61B 5/097; A61B 5/00

(52) U.S. Cl. CPC A61B 5/097; A61B 5/002; A61B 2562/12; A61B 2562/0271; A61B 5/0022

(57) ABSTRACT

The present invention is a breath sample collecting mask comprising a mask body and a breath sample collector that provides non-invasive sample collection for detection of infectious diseases as well as lung and respiratory diseases, potentially before the onset of symptoms. The breath sample collector contains a filter that catches viral, bacterial, and other biomarker particles. The breath sample collector can be removed from the mask and placed into a tube for sample elution and further processing and analysis. The breath sample collecting mask may further comprise a thermometer, electrodes attached on the filter of the sample collector, and a microprocessor to monitor the mask wearer's condition.

### CROSS-REFERENCE TO RELATED APPLICATIONS

This application claims priority under 35 U.S.C. § 119(e) to U.S. Provisional Application Ser. No. 63/001,103, titled “APPARATUS FOR BREATH SAMPLE COLLECTION,” filed on Mar. 27, 2020, which is hereby incorporated herein by reference in its entirety.

### BACKGROUND

### Field of Invention

The present disclosure is directed to an apparatus for collecting breath samples and for early detection of infectious diseases as well as lung and respiratory diseases. More particularly, the present disclosure provides methods and designs of a breath sample collecting mask with or without an exhalation valve and a breath sample collector for capturing viral and bacterial particles as well as biomarkers of lung and respiratory diseases from one's breath for further processing and analysis. It can also be used for capturing molecules of an illicit drug from one's breath.

### Description of Prior Art

The disease caused by the novel coronavirus, known as COVID-19, has been rapidly spreading around the world. Real-time reverse transcriptase assays (RT-PCR) are recommended to diagnose COVID-19 infections by looking for viral RNA or DNA sequences. Lateral Flow Assays (LFA) are also used for rapid diagnostic testing by using antibodies to detect antigen structures found on the surface of pathogens. Recent research shows positive results for RT-PCR assays a day before the onset of symptoms, and the viral loads in throat swab and sputum samples peaked at a range of around 104 to 107 copies per mL at around 5 to 6 days after the onset of symptoms, suggesting that infected patients can be infectious before they become symptomatic. A German-based research team showed that some people with COVID-19 had high levels of the virus detected in throat swabs early in their illness, when their symptoms were mild. This means the pathogen could easily be released through coughs or sneezes—a process known as viral shedding—and spread to others. Other research shows that the pathogen responsible for the COVID-19 pandemic has an incubation period that is approximately five days shorter than when patients first show symptoms. The spread during this period when a patient is unaware of his or her infection is called “cryptic transmission” and it causes the airborne virus or bacteria to spread to a greater extent than realized. This is extremely worrisome for the spread of this virus. Therefore, early detection of pathogens before the onset of symptoms is crucially important to cut off the pandemic.

Collecting a sample from respiratory passages, such as one's nasal cavity or throat, for the detection of infection diseases presents many challenges: 1) collecting samples from a nasal cavity, throat, or other respiratory passages is difficult; 2) the sampling position and/or sampling amount may be off and the resulting sample may be inaccurate, causing a false positive; 3) the operation needs to be performed in a hospital or in a clinic by medical professionals; 4) cross-infection may occur during sample collection.

Thus, a system is needed to provide non-invasive and/or non-intrusive sample collection. It should allow sample collection to be consistent and efficient. It should be user-friendly both for professionals and patients. Furthermore, the system should be compatible with rapid test kits for point-of-care application and for self-testing at home, in order to detect the virus before the onset of any symptoms.

### BRIEF SUMMARY OF THE INVENTION

The present invention's unique design overcomes the aforementioned limitations and offers an easy, user-friendly, and non-location dependent way to detect viruses, pathogens, and biomarkers, potentially before the onset of symptoms.

One embodiment of the present invention provides a breath sample collecting mask comprising a mask body and a breath sample collector, wherein the breath sample collector is a cartridge with an embedded filter. The cartridge may comprise a plastic frame, a side of Velcro, or a piece of double-sided tape and the filter may comprise a highly breathable and thermal comfort filter. The cartridge may be removed after the breath sample collecting mask is used. The mask comprises valves that do not allow contaminants to contaminate the breath sample collector. This design allows breath samples to be easily collected regardless of the mask user's location.

The present invention may further comprise a tube to collect the cartridge once it is removed. The tube has a flat, rectangular shape and may have a funnel at one end. The funnel may be attached to a funnel adapter, wherein the funnel adapter is directly connectable to a test device or test equipment. This functionality further reduces the need for the mask user to be in a specific location or for the mask user to require the assistance of a medical professional for breath sample collection.

The present invention may further comprise a temperature sensor and a microprocessor. These components aid in the determination of the user's condition. The microprocessor may be able to calculate the wearer's body temperature based on the wearer's breath temperature. The sensor and the microprocessor may also monitor the condition of the mask-wear. The microprocessor may be a Bluetooth module that communicates with compatible devices including smartphones, to display the temperature, breathing rate, and condition of the mask-wearer.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings, where:

FIG. 1 is a perspective view of one embodiment of an exhalation valve with a breath sample collector on a breath sample collecting mask.

FIG. 2 is a front view of the breath sample collecting mask in FIG. 1.

FIG. 3 is a detailed inside view of the breath sample collector on the breath sample collecting mask in FIG. 1.

FIG. 4 is a detailed front view of the exhalation valve on the breath sample collecting mask in FIG. 1.

FIG. 5 is a sectional view of the exhalation valve and the breath sample collector on the breath sample collecting mask in FIG. 1, wherein the exhalation valve is closed.

FIG. 6 is a sectional view of the exhalation valve and the breath sample collector on the breath sample collecting mask in FIG. 1, wherein the exhalation valve is open.

FIG. 7 is a front view of the breath sample collector in FIG. 1.

FIG. 8 is a back view of the breath sample collector in FIG. 1.

FIG. 9 is a front view of another embodiment of a breath sample collector.

FIG. 10 is a back view of the breath sample collector in FIG. 9.

FIG. 11 is an inside view of another embodiment of a breath sample collecting mask with the breath sample collector in FIG. 9.

FIG. 12 is a detailed inside view of the breath sample collecting mask in FIG. 11 with a loop side of Velcro for sticking the breath sample collector in FIG. 9.

FIG. 13 shows a tube for containing the breath sample collector for a rapid test kit, or for shipping to a lab for further testing.

FIG. 14 shows a sectional view of an embodiment of a breath sample collecting mask with an exhalation valve, a breath sample collector, and a temperature sensor.

FIG. 15 is a front view of the breath sample collector in FIG. 14.

### DETAILED DESCRIPTION OF THE INVENTION

The terminology used herein is for the purpose of describing particular embodiments only and is not intended to be limiting of the invention. As used herein, the terms “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning in the context of relevant art and the present disclosure will not be interpreted in an idealized or overly formal sense unless expressly so defined herein.

In describing the invention, it will be understood that a number of elements and techniques are disclosed. Each of these has individual benefit, and each can also be used in conjunction with one or more, or in some cases all, of the other disclosed elements and techniques. Accordingly, for the sake of clarity, this description will refrain from repeating every possible combination of the individual elements or techniques in an unnecessary fashion. Nevertheless, the specification and claims should be read with the understanding that such combinations are entirely within the scope of the invention and the claim.

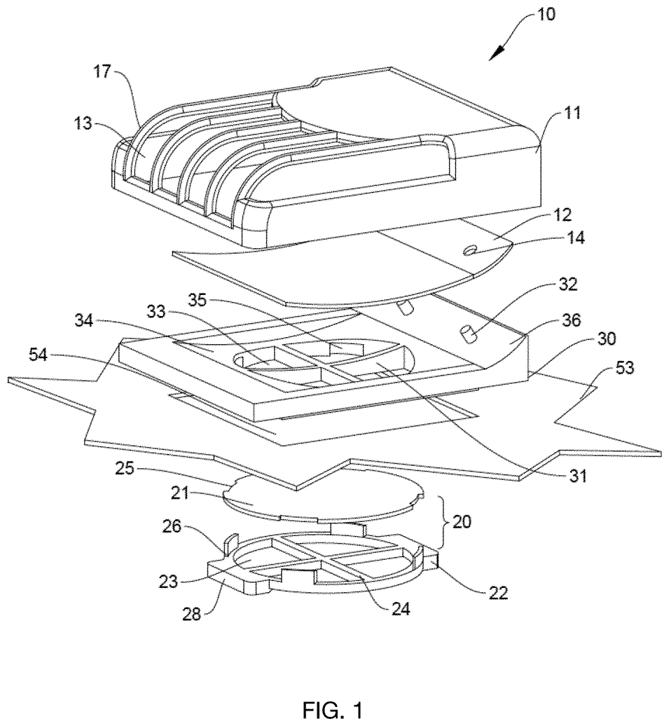

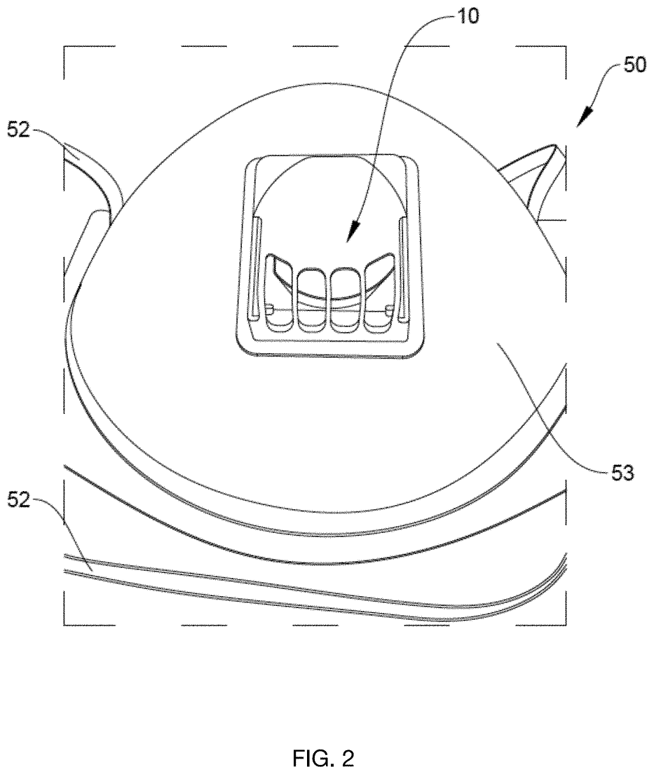

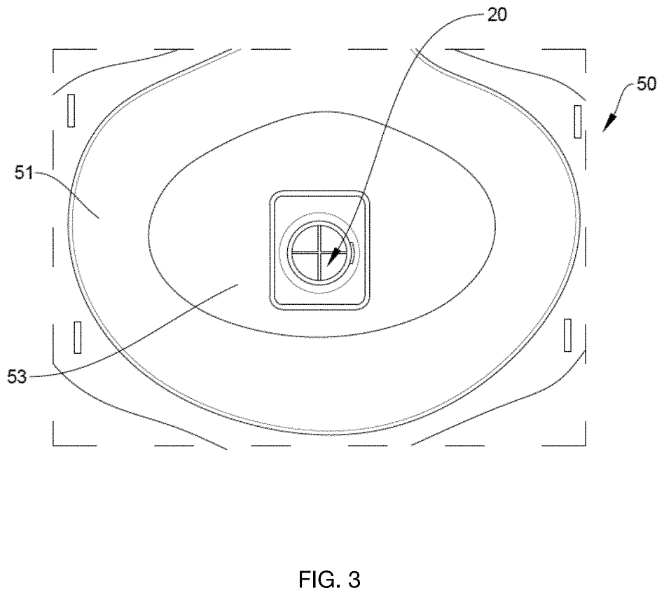

As illustrated in FIG. 1 and FIG. 2, the breath sample collecting mask 50 may comprise an exhalation valve 10 with a breath sample collector 20. The breath sample collecting mask 50 may have a cup-shaped mask body 53 and rubber straps or tie strings 52. The mask body 53 may provide an opening 54 through which an exhalation valve seat 30 is mounted on. The breath sample collector 20 may comprise a porous polymer filter 21 and a cartridge 22 and may be attached to and detached from the back side of the valve seat 30, as illustrated in FIG. 3 inside of the mask body 53.

As illustrated in FIG. 1, the valve seat 30 may have a seal surface 34 that may contact a flexible flap 12 when air is not flowing through the valve 10. An orifice 33 may be located radially inward of the seal surface 34 and may have a cross support 31 that prevents the flexible flap 12 from inverting into the orifice 33 during reverse air flow during, for example, inhalation. The valve seat 30 can be secured to the mask body 53 by ultrasonic welding, an adhesive, or other suitable means.

As illustrated in FIG. 1, the breath sample collector 20 may have an orifice 23 and the orifice 23 may have a cross support 24 that prevents the porous polymer filter 21 from inverting into the orifice 23. The cross 31 on the valve seat 30 may prevent the porous polymer filter 21 from inverting outwards into the orifice 33 during exhalation. The porous polymer filter 21 may have notches 25. On the cartridge 22, there may be four buckles 26 corresponding to the four notches 25 on the porous polymer filter 21 that hold the cartridge 22 and the porous polymer filter 21 together. The valve seat 30 may have four notches 35 and the four notches 35 on the valve seat 30 that hold the breath sample collector 20 on the valve seat 30 inside of the breath sample collecting mask 50. This configuration may allow the breath sample collector 20 to be easily removed from the valve seat 30 by pulling one of the handle bars 28 after the breath sample is taken.

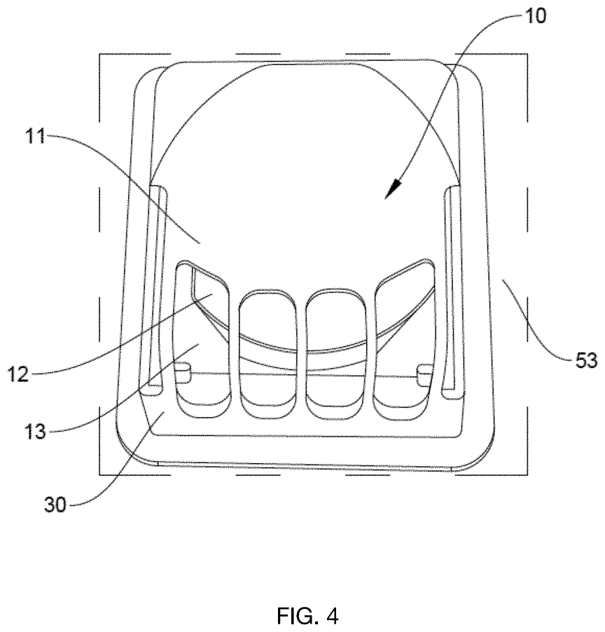

As illustrated in FIG. 1 and FIG. 4, the exhalation valve 10 may have a valve cover 11 to protect the flexible flap 12, and to help prevent the passage of contaminants through the exhalation valve 10. The valve cover 11 may be secured to the valve seat 30 by ultrasonic welding, adhesives, or other common chemical or physical means. The valve cover 11 may have openings 13 for the passage of air flow when a wearer exhales. The valve cover may be partially comprised of ribs 17 for further structural support and aesthetics.

The exhalation valve 10 on the mask body 53 may be positioned directly in front of the wearer's mouth and nose, where viral shedding, bacteria, and any biomarker particles are released through coughs or sneezes, is captured by the porous polymer filter 21 on the breath sample collector 20. The porous polymer filter 21 may be made from an electret material. The porous polymer filter 21 may capture, through adherence or interception, any virus or bacteria particles, or any biomarkers, as well as any drug compositions larger than 1 μm across. In other embodiments, the porous polymer filter 21 may capture, through adherence or interception, any virus or bacteria particles, or any biomarkers, as well as any drug compositions larger than a specified size selected for certain applications. The virus or bacteria or other biomarker particles, as well as any drug compositions that are smaller than 1 μm may be captured by the electrostatic forces of the porous polymer filter. The exhalation valve 10 may close during inhalation, preventing the breath sample collector 20 from contacting outside particles because the flexible flap 12 of the exhalation valve 10 may be in the front of the breath sample collector 20.

The porous polymer filter 21 on the breath sample collector 20 may be a highly breathable and thermal comfort filter comprising an electret polyethersulfone barium nanofibrous membrane or an electret titanate nanofibrous membrane (PES/BaTiO3 NFM), wherein the membrane may be integrated on a nonwoven polypropylene substrate. Benefiting from its high porosity and optimized injection charge energy, this membrane achieves a collection efficiency of 99% for the particle diameters in the range of 0.5 to 20 μm. The membrane also possesses a good air permeability of 743 mm/s, a modest water vapor permeability of 6.24 kg/m2/d, and an enhanced charge storage stability.

The mask body 53 may be fluid permeable to inhaled air. The mask body may be cup-shaped, and may also be curved, hemispherical-shaped, or may be any other shape that is preformed into a desired face-fitting configuration that retains its configuration during use and provides enough distance between the cartridge 22 on the breath sample collector 20 to the nose or the mouth of the wearer to allow the wearer to speak or breathe freely.

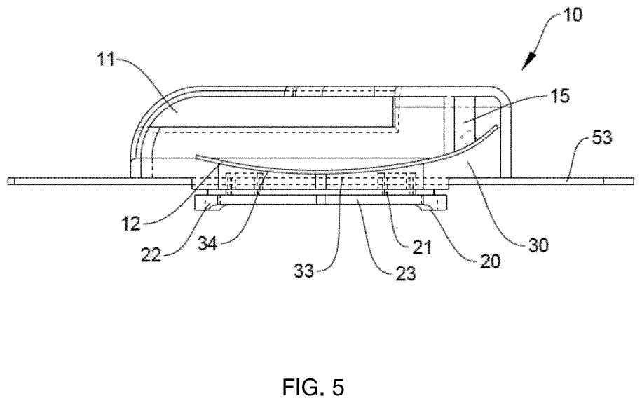

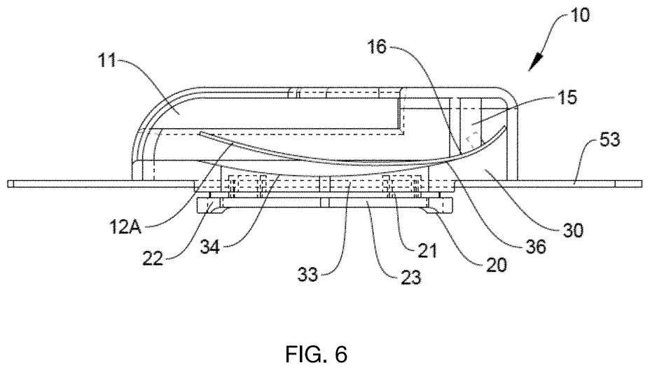

As illustrated in FIG. 1, FIG. 5, and FIG. 6, the seal surface 34 may be rectangular and the orifice 33 may be circular. The seal surface 34 and the orifice 33 may take on any shape including, but not limited to, square, rectangular, circular, or elliptical. The shape of seal surface 34 may or may not correspond to the shape of orifice 33. It is only necessary that the seal surface 34 circumscribes the orifice 33 and the flexible flap 12 covers the orifice 33.

FIG. 5. illustrates the flexible flap 12 in a closed position. FIG. 6. Illustrates the flexible flap 12 in an open position. The seal surface 34 may have a concave curvature as illustrated in the sectional view of FIG. 5 and FIG. 6. This concave curvature of the seal surface 33 may correspond to the deformation curve of the flexible flap 12 that may be secured by a cantilever beam 15 on the valve cover 11 and a flap retaining surface 36 on the valve seat 30. As illustrated in FIG. 5, during inhalation, the outside pressure is higher than the pressure inside of the mask 50, exerting an inward pressure on the flexible flap 12, causing the flexible flap 12 to contact the seal surface 34 and closing the valve 10 to prevent the undesired influx of contaminates through the orifice 33 to the breath sample collector 20. As illustrated in FIG. 6, during exhalation, the inside pressure is higher than the pressure outside of the mask 50, causing a certain amount of air flow from the wearer's mouth and nose to pass through the orifice 33, exerting a pressure on the flexible flap 12A, causing its free end to be lifted from the seal surface 34 of the valve seat 30, causing the valve 10 to open.

As illustrated in FIG. 1, FIG. 5, and FIG. 6, the flexible flap 12 may be pressed by a flap retaining surface 16 on the bottom of the cantilever beam 15 onto the flap retaining surface 36 on the valve seat 30 that retains the deformation curve of the flexible flap 12, sealing the orifice 32. On the flap retaining surface 36, pins 32 may be positioned corresponding to the holes 14 on the flexible flap 12 to hold the flexible flap 12 in place. The flexible flap 12 may also be attached to the flap retaining surface 36 by ultrasonic welding, adhesives, or other common chemical or physical means.

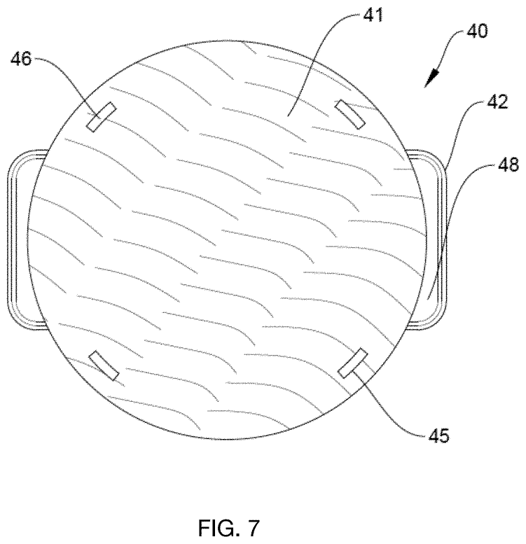

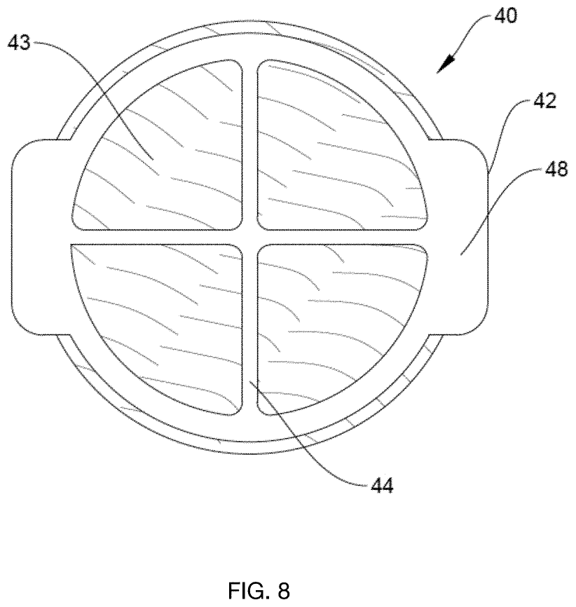

FIG. 7 and FIG. 8 illustrate another embodiment of the assembled breath sample collector 40 with its porous polymer filter 41 and its cartridge 42. The four buckles 46 may hold the porous polymer filter 41 on the cartridge 42 through the four slots 45 on the porous polymer filter 41. The breath sample collector may have orifices 43 and a cross support 44. To mount the porous polymer filter 41 onto the cartridge 42, common chemical or physical means that do not inhibit the breath sample collector's functionality may be used, including, but not limited to, ultrasonic welding and heat clamping. Adhesives and such should not be used because they may release chemicals that interfere with the detection of pathogens. The shape of the breath sample collector 40 may be circular with a handle bar 48 on both sides. The breath sample collector may take on any other shape that does not inhibit its functionality including, but not limited to, square, rectangular, or elliptical.

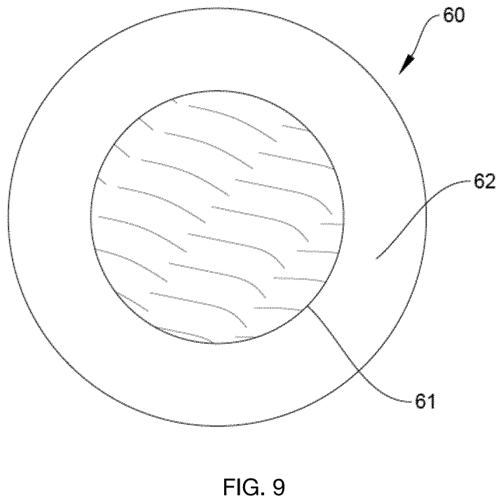

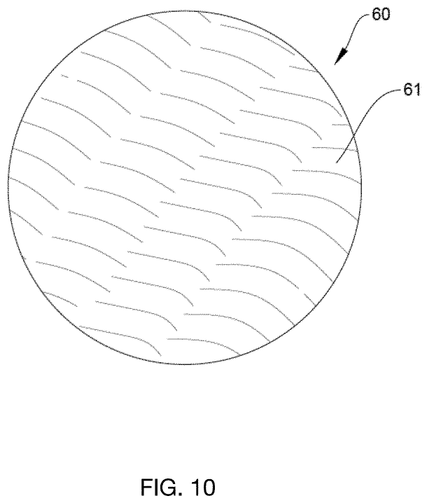

FIG. 9 and FIG. 10 illustrate another embodiment of the assembled breath sample collector 60 comprising a porous polymer filter 61 on the assembled breath sample collector's frame 62. The ring-shaped frame 62 may be a hook side of Velcro. The porous polymer filter 61 may be sewn into the frame 62, but ultrasonic welding, mechanical clamping, or other chemical or physical means that do not inhibit the breath sample collecting mask's functionality may be used. Adhesives and such should not be used because they may release chemicals that interfere with the detection of pathogens. The shape of the breath sample collector 60 may be circular with the ring-shaped frame 62. The breath sample collector may also take on any other shape that does not interfere with the breath sample collector's functionality such as square, rectangular, or elliptical.

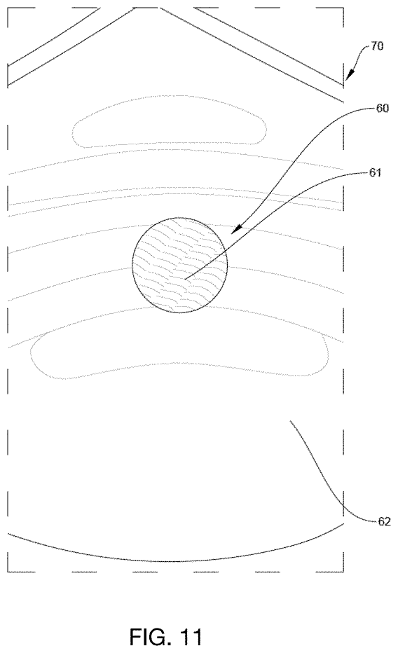

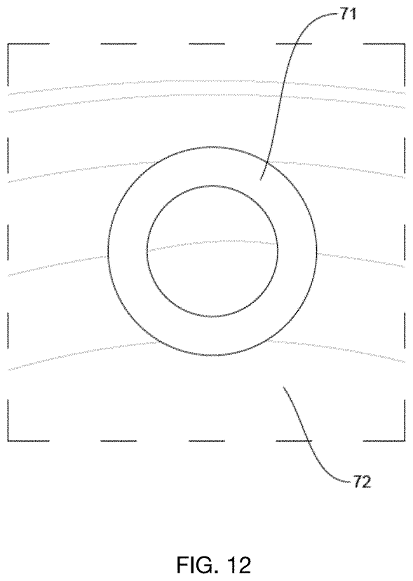

FIG. 11 and FIG. 12 illustrate another embodiment of the breath sample collecting mask 70 comprising the breath sample collector 60. The breath sample collecting mask 70 may be a regular filtering mask with a cup-shaped mask body 72. Inside of the mask 70, at the front of wearer's mouth and nose, a piece of loop side Velcro 71 may be mounted by ultrasonic welding, adhesives, mechanical clamping, or other chemical or physical means that do not inhibit the breath sample collecting mask's functionality. The piece of loop side Velcro 71 may be ring shaped as illustrated in FIG. 12. The piece of loop side Velcro 71 may also be any other shape that does not inhibit the breath sample collecting mask's functionality including, but not limited to, square, rectangular, or elliptical. The assembled breath sample collector 60 may be attached on the inside of the breath sample collecting mask 70 with the hook side Velcro frame 62 sticking to the loop side Velcro 71 so that the breath sample collector 60 is attachable to the inside of the filtering face mask 70 as illustrated in FIG. 11.

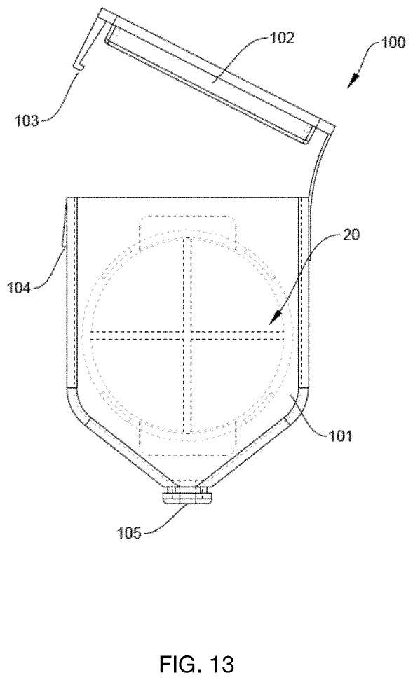

After the breath sample is collected, the breath sample collector embodiments 20, 40, or 60 may be removed from the valve seat 30 or the Velcro frame 71, and placed into a tube 100, as illustrated in FIG. 13. The tube 100 may comprise a flat rectangular tubular section 101 and a lid section 102. The flat rectangular tubular section 101 may hold within it a breath sample collector 20 40, or 60. The tube 100 may be sealed with the lid 102 and locked with an elbow latch 103 and 104. The tube 100 with the breath sample collector 20, 40, or 60 inside, may be shipped to an analytic laboratory to test the breath sample. For point-of-care testing, a buffer solution may be loaded in the tube 100 to wash off pathogens captured on the porous polymer filter 21, 41, or 61 for RT-PCR, RNA, DNA, or LFA protein tests.

A funnel adaptor 105 may be attached to the bottom of the tube 100 and the funnel adaptor 105 may be directly connected to an LFA test strip for liquid transferring, eliminating the need to use a pipette for moving the liquid that is the buffer containing the pathogens washed off of the breath sample collector 20, 40, or 60. The funnel adaptor 105 may also be used to connect to test devices or equipment directly.

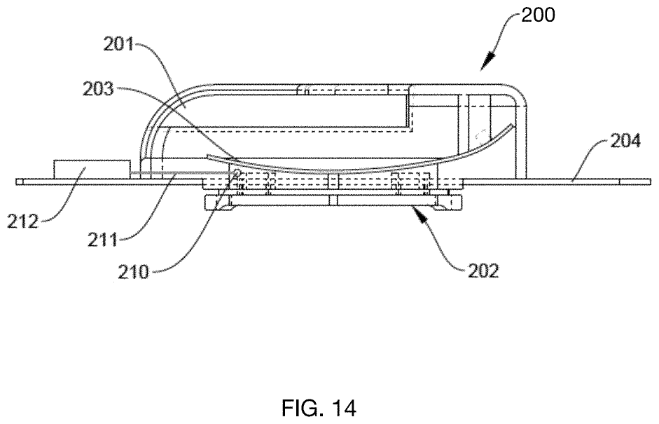

FIG. 14 illustrates an embodiment of a breath sample collecting mask 200 comprising an exhalation valve 201 and a breath sample collector 202, that is similar to the embodiment of the breath sample collecting mask 50 and the exhalation valve 10. A temperature sensor 210 may be placed in the breath pathway between the sample collector 202 and the flexible flap 203. The temperature sensor 210 may be connected to a microprocessor module 212 through a wire 211. The sensor 210 may capture the wearer's breath temperature from exhalation. The microprocessor 212 may be able to calculate the wearer's body temperature based on the wearer's breath temperature. The sensor 210 and the microprocessor 212 may also monitor the condition of the mask-wearer. The microprocessor may be a Bluetooth or WiFi module that communicates with compatible smart devices including smartphones or tablet, to display the temperature, breathing rate, and condition of the mask-wearer. Furthermore, the smart device may be used to measure the test results quantitatively or detect the test results qualitatively of the sample collected by the breath sample collector 202.

The temperature sensor 210 and the microprocessor 212 may be embedded in the body of the exhalation valve 201 or the body 204 of the face mask 200, or may be a separate device, such as an earbud. The temperature sensor 210 may be placed at the outlet of the exhalation valve, and the temperature sensor 210 may be connected to a microprocessor plugged into the ear.

The temperature sensor 210 and the microprocessor 212 may also to be used with a disposable breath sample collecting mask such as the breath sample collecting mask 60 with the breath sample collector 40, wherein the microprocessor 212 may be a separated device, and the temperature sensor 210 may be embedded in the body of the mask at the front of the mouth, and wherein the microprocessor 212 and the sensor 210 are connected through a wire 211 and a plug.

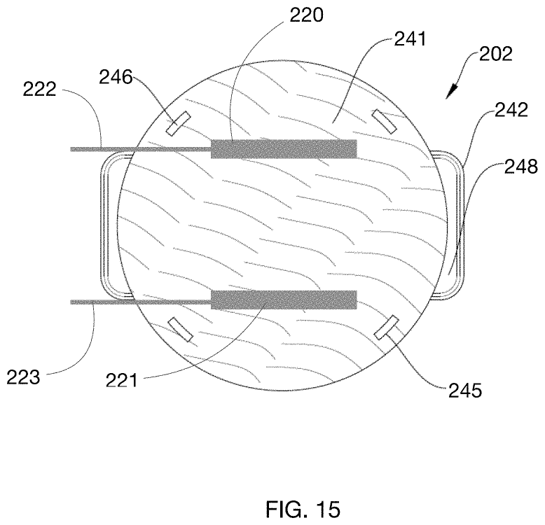

FIG. 15 illustrates an embodiment of the breath sample collector 202 with its porous polymer filter 241 and its cartridge 242. The four buckles 246 may hold the porous polymer filter 241 on the cartridge 242 through the four slots 245 on the porous polymer filter 241. The breath sample collector 202 may be made similar to the breath sample collector 40 with orifices and cross support on its back. The shape of the breath sample collector 202 with a handle bar 248 on both sides may be circular but may be any other shape that does not inhibit its functionality, including, but not limited to, square, rectangular, or elliptical. On the porous polymer filter 241, two electrodes 220 and 221 are attached or embedded. The shape of the two electrodes 220 and 221 may be rectangular but may be any other shape that does not inhibit its functionality, including, but not limited to, square, or concentric circular or elliptical. The electrode 220 and 221 connect to the microprocessor 212 through two wires 222 and 223 and maybe other circuits to detect the moisture on the filter 241. The electrode 220 and 221 may apply a voltage to the filter 241 that may generate or increase the charge of the filter 241 to capture virus or bacteria, or other biomarker particles, as well as any drug compositions by the electrostatic force. The electrode 220 and 221 may be made from sheets of stainless steel foil or by depositing or coating Ag/AgCl, carbon, or other conductive material.

### One Variation of Breath Sample Collection

When a wearer of the filtering face mask 50 inhales, the exhalation valve 10 closes and no contaminants from outside are able to contaminate the porous polymer filter 21 on the breath sample collector 20. Outside air passes through the filtering material of the mask body 53. Virus, bacteria, and other particles are blocked by the filtering material.

When a wearer of a filtering face mask 50 exhales, as the inside pressure is higher than the outside pressure, the exhalation valve 10 opens. Exhaled air passes through the mask body 53 and the porous polymer filter 21 on the breath sample collector 20. As the porous polymer filter 21 is on the front of the wearer's mouth and nose, most of the virus, bacteria or other biomarker particles released through coughs or sneezes may be captured by the porous polymer filter 21. To collect a quality breath sample, the wearer should have the mask 50 on for a certain time, for example, 4 hours. After the breath sample collecting mask 50 is taken off, the breath sample collector 20 may be removed and placed in the tube 100, and sealed by closing the lid 102.

Although this invention herein has been described with reference to the particular embodiments, it is to be understood that these embodiments are merely illustrative of certain principles and applications of the present invention. Numerous modifications may be made to the illustrative embodiments and other arrangements may be devised without departing from the spirit and scope of the present invention as defined by the following claims.

What is claimed is:

1. A breath sample collecting mask comprising a mask body and a breath sample collector placed within the mask body in front of a mouth or nose of a wearer, wherein the breath sample collector comprises a porous polymer filter and a cartridge, and wherein the porous polymer filter is fixed on the cartridge.

2. The breath sample collecting mask as in claim 1, wherein the cartridge is removable from the mask body.

3. The breath sample collecting mask as in claim 2, wherein the cartridge is a plastic frame, a side of Velcro, or a piece of double-sided tape.

4. The breath sample collecting mask as in claim 3, wherein the plastic frame of the cartridge is detachable onto an exhalation valve seat, wherein the exhalation valve seat is secured to the mask body through ultrasonic welding, adhesives, or other chemical or physical means that do not inhibit the breath sample collecting mask's functionality.

5. The breath sample collecting mask as in claim 3, wherein an opposing side of Velcro is mounted on an inside of the mask body, and wherein the side of Velcro that comprises the cartridge is stuck to the opposing side of Velcro mounted on the inside of the mask body.

6. The breath sample collecting mask as in claim 3, wherein the cartridge is attached to the inside of the mask body using the piece of double-sided tape, wherein the double-sided tape does not adhere to the breath sample collector when the breath sample collector is removed from the mask body.

7. The breath sample collector as in claim 1, wherein the porous polymer filter is a highly breathable and thermal comfort filter.

8. The porous polymer filter as in claim 7, wherein the highly breathable and thermal comfort filter is an electret polyethersulfone nanofibrous membrane or a barium titanate nanofibrous membrane (PES/BaTiO3 NFM) integrated onto a nonwoven polypropylene substrate.

9. The breath sample collector as in claim 1, wherein the porous polymer filter is square, rectangular, circular, elliptical, or an irregular shape, and wherein the porous polymer filter has an area between 1 square centimeter and 25 square centimeters.

10. The breath sample collecting mask as in claim 1, wherein the mask body is cup-shaped, curved, hemispherical-shaped, or has any shape that is preformed into a desired face-fitting configuration that retains that configuration during use, wherein the distance between the breath sample collector to the mouth or nose of the wearer is retained, allowing the wearer to speak and breathe freely.

11. The breath sample collecting mask as in claim 1, further comprising a tube, wherein the tube has a flat rectangular shape with a funnel at one end.

12. The tube as in claim 11, wherein the funnel is attached to a funnel adapter, wherein the funnel adapter is directly connectable to a test device or test equipment.

13. A breath sample collecting mask comprising a mask body, a breath sample collector placed within the mask body in front of a mouth or nose of a wearer, a temperature sensor, and a microprocessor, wherein the breath sample collector comprises a porous polymer filter and a cartridge, and wherein the porous polymer filter is fixed on the cartridge.

14. The breath sample collecting mask as in claim 13, wherein the temperature sensor is placed between the breath sample collector and the mask body.

15. The breath sample collecting mask as in claim 13, wherein the microprocessor and the temperature sensor are embedded in the mask body.

16. The breath sample collecting mask as in claim 13, wherein the microprocessor is attached to the outside of the breath sample collecting mask, and wherein the microprocessor is connected to the temperature sensor.

17. The breath sample collecting mask as in claim 13, wherein the microprocessor is a separate device, and wherein the separate device is a Bluetooth module or a WiFi module.

18. The breath sample collecting mask as in claim 13, wherein on the breath sample collector electrodes are attached or embedded, and wherein the electrodes connect the microprocessor through wires and maybe other circuits.

19. The breath sample collector as in claim 18, wherein the electrodes are in the shape of rectangular, square, or concentric circular or elliptical.

20. The breath sample collector as in claim 18, wherein the electrodes are made from sheets of stainless steel foil or by depositing or coating Ag/AgCl, carbon, or other conductive material.
